(19) United States
(12) Patent Application Publication
(10) Pub. No.: US 2018/0296104 A1
(43) Pub. Date: Oct. 18, 2018

(54) NON-INVASIVE BLOOD PRESSURE MEASUREMENT

(71) Applicant: AtCor Medical Pty Ltd, West Ryde (AU)

(72) Inventor: Ahmad M. Qasem, Guildford, NSW (AU)

(73) Assignee: AtCor Medical Pty Ltd, West Ryde (AU)

(21) Appl. No.: 15/943,970

(22) Filed: Apr. 3, 2018

Related U.S. Application Data
(60) Provisional application No. 62/485,128, filed on Apr. 13, 2017.

(51) Int. Cl. A61B 5/021; A61B 5/00

(52) U.S. Cl. CPC A61B 5/02116; A61B 5/7282; A61B 5/7203; A61B 5/7225; A61B 5/7285

(57) ABSTRACT

A method of measuring a patient's blood pressure non-invasively considers the shape of the waveform to accurately estimate the patient's invasive systolic and diastolic blood pressure, or alternatively accurately predict the patient's hypertension classification. The method can be implemented in a clinical setting or within a wearable device.

### FIELD OF THE INVENTION

The invention pertains to measuring systolic and diastolic blood pressure non-invasively, without using a brachial cuff operating in oscillometric mode. The invention is directed to calibrating a non-invasive arterial pulse waveform based on the shape of a scaled version of the waveform so that its maximum and minimum values accurately estimate the patient's systolic (SP) and diastolic blood pressure (DP). Alternatively, instead of determining SP and DP, the invention determines a clinical classification for which the patient's SP and DP are expected to qualify, such as optimal, normal, high normal, and grade of hypertension.

### BACKGROUND OF THE INVENTION

Arterial blood pressure is a clinically important indicator of the status of the cardiovascular system, reflective of arterial and cardiac load and an early independent predictive marker of cardiovascular events and diseases. However, to measure the inter-arterial blood pressure accurately requires an invasive procedure to insert a catheter with a pressure sensor inside the artery. As a result, non-invasive methods were created to estimate pressure at the peripheral brachial artery.

One of the earliest non-invasive methods to estimate pressure in the brachial artery is the auscultatory method which requires inflating a cuff wrapped around the patient's upper arm and brachial artery until the brachial artery occludes (i.e., no blood flow). Then, the cuff is gradually deflated and blood starts flowing with “thumping” sounds that can be detected through a stethoscope. The first “thumping” sound should occur when the cuff pressure equals the patient's systolic pressure (maximum pressure during cardiac ejection) and the last “thumping” sound should occur when the cuff pressure equals the patient's diastolic pressure (minimum pressure during cardiac filling).

For decades, the auscultatory method was used for clinical hypertension diagnosis and had become the standard for non-invasive blood pressure measurement. However, the accuracy of the measured pressure value was dependent on the operator's acute detection of the heart sound and also dependent on the rate that the operator deflates the cuff. Because the accuracy of the auscultatory method is operator dependent, an automated method was established based on detecting oscillatory pulsations measured by the brachial cuff during cuff inflation or deflation. The height of the pulse oscillation increases when the cuff pressure decreases from systolic pressure to below systolic pressure and the height of the oscillation decreases when the cuff pressure decreases from above diastolic pressure to diastolic pressure and below. Based on this concept, current “oscillometric” devices apply different algorithms to detect oscillation heights related to systolic and diastolic pressure.

Oscillometric cuff devices are often called non-invasive blood pressure devices or NIBP devices in the art. To be accepted for clinical use, an NIBP device has to show equivalence to the standard auscultatory method based on the American National Standard for Non-Invasive Automated Blood Pressure Devices, ANSI/AAMI/ISO 81060-2:2009, “Non-invasive sphygmomanometers—Part 2: Clinical validation of automated measurement type,” Section 5.2.4.1.2 Part a—Criterion 1, page 20 (which states that the mean error for determination of all subjects in the test “shall not be greater than 5.0 mmHg with a standard deviation no greater than 8 mmHg.”) Accordingly, any oscillometric cuff device can pass the validation requirements if the average difference with the auscultatory method for systolic and diastolic pressure is not more than 5 mmHg and the standard deviation is not more than 8 mmHg. This means that approved oscillometric devices can register a difference with the standard auscultatory method reaching above 20 mmHg for some data points.

Oscillometric automated blood pressure devices have been standard in clinical practice for many years, and have also been used in medical research to assess cardiovascular risk. Even though non-invasive blood pressure (NIBP) measurement identifies a percentage of the general population at risk of cardiovascular diseases, a large group is not identified by NIBP measurement to be at risk even though they may be at risk. The main reason is that measured blood pressure varies among different NIBP devices due to the different devices having different propriety algorithms for detecting systolic and diastolic pressure. Furthermore, when compared to invasive pressure values, NIBP devices have been shown to underestimate systolic pressure and overestimate diastolic pressure, see, Sharman et al., “Validation of non-invasive central blood pressure devices: Artery Society task force consensus statement on protocol standardization”, European Heart Journal (2017) 0, 1-10; Cloud et al., “Estimation of central aortic pressure by SphygmoCor® requires intra-arterial peripheral”, Clinical Science (2003) 105, 219-225; Shoji et al., “Invasive validation of a novel brachial cuff-based oscillometric device (SphygmoCorXCEL) for measuring central blood pressure”, Journal of Hypertension 2016, 34. Accordingly, since measuring brachial pressure invasively is the gold standard, non-invasive measurements that closer estimate the invasive pressure and overcome the errors inherent in cuff NIBP devices would be a significant improvement in the field of blood pressure measurement and its clinical importance.

First, as mentioned, with the maximum acceptable error standard deviation (see ANSI/AAMI/ISO 81060-2:2009, “Non-invasive sphygmomanometers—Part 2: Clinical validation of automated measurement type”, Section 5.2.4.1.2 Part a—Criterion 1, page 20) being 8 mmHg for a statistically approved NIBP cuff device, the device may have an error of 10 mmHg or above on about 20-30% of the general population. This relatively high margin of error means that some subjects with cardiovascular risk are classified as healthy and some are classified as healthy when they should in fact be classified as at risk.

Second, invasive pressure data has shown that the difference between cuff NIBP and invasive brachial artery SP and DP typically has either a high average error or high error standard deviation that would exceed 15 mmHg on a large percentage of the study population (see, Cloud et al. and Shoji et al. referenced above). These errors reduce NIBP reliability significantly in clinical practice.

Third, different cuff NIBP devices use different algorithms to detect SP and DP from cuff oscillatory pulses, which results in variations between the NIBP devices' measurements adding to cuff NIBP unreliability.

Fourth, given that blood pressure and heart rate continuously adjust based on the body's demand due to metabolism, blood pressure and heart rate are not constant and can change from beat to beat. The continuous monitoring of beat-to-beat blood pressure, like heart rate with ECG devices, would provide a useful blood pressure variability assessment tool, such as the ability to immediately detect sudden changes in blood pressure that allows prompt medical staff response. Like heart rate monitors providing an ECG, devices monitoring beat-to-beat blood pressure will be clinically valuable. Yet, the cuff NIBP measurements, which take about 30 seconds to 2 minutes to measure SP and DP, do not measure blood pressure continuously beat by beat. Furthermore, blood pressure may change during the cuff NIBP duration of blood pressure measurements producing inaccurate blood pressure values.

Fifth, the oscillometric cuff NIBP devices require the cuff to be inflated above SP occluding the brachial artery and seizing blood flow for few moments which may cause patient's discomfort. Even though the cuff NIBP devices are low risk devices, such inconvenience may also affect blood pressure which the device is trying to measure.

As a result, attempts have been made to estimate SP and DP without using cuff NIBP in order to provide continuous blood pressure measurements without the inconvenience of a cuff obstructing and disturbing brachial arterial blood flow. One of the most common methods (Masé et al., Journal of Electrocardiology 2011-44 pp 201-207; Chen et al., Annals of Biomedical Engineering 2012, Vol. 40, No. 4, pp. 871-882; Zheng et al., J Med Syst 2016, 40:195; Fuke et al., Zheng et al., and Sola et al., 35th Annual International Conference of the IEEE EMBS 2013 July) is detecting SP and DP by measuring the pulse wave velocity (PWV) or pulse transit time (PTT) between two simultaneously measured arterial pulses or between a simultaneously measured ECG signal and an arterial pulse. These methods are based on the fact that pulse wave velocity, which is calculated from PTT, is related to pressure. Accordingly, by measuring PTT, blood pressure can be estimated or detected. However, the method requires calibration with a cuff NIBP device for the first PTT measurement on any setting, like a different patient or different patient's posture, because the relationship between PTT and blood pressure is related to change. After calibration, the initial PTT is associated with SP and DP values and any changes in PTT afterward relate to changes in blood pressure. The method still requires a cuff NIBP every time it is used in different settings, like for a different patient or different patient's posture, which means the method is not totally cuff-less. Another issue with the method is that it requires simultaneous recordings of two signals at different positions, which adds complication in the hardware design to assure accuracy of the recordings let alone the inconvenience of having sensors at two arterial locations.

Another method was proposed by Baruch (U.S. Pat. No. 8,100,835 B2) to estimate SP and DP from one arterial pulse recording. The method consisted of decomposing and then identifying three (3) peaks from a recorded radial pulse. The method relates the time between the peaks with SP and DP. Implementing such a method faces the same issue faced with the PTT methods, namely, the need for calibration or individualizing the method. The method by Baruch identified that the linear relationship between the time between the peaks in the arterial pulse recording and SP and DP is different between different subjects in the population. The solution according to Baruch is to have different linear relationships based on gender, height, disease status, fitness or/and any other parameters in the patient's profile. Individualizing the method this way is impractical and renders the detection of SP and DP from a pulse redundant because the patient's profile will be the main determents of SP and DP.

Another method by Lading et al. (U.S. Patent Application US 2015/0327786 A1) estimates pulse pressure PP, which is equal to SP-DP, and mean pressure from changes in the cross sectional area distension related to the pulse in a peripheral artery (e.g., brachial, radial or finger). The method first requires recording of two measurements of the peripheral arterial distension pulse at different hydrostatic pressures (hand down and hand raised at the heart level) to determine the relationship between the recorded changes in arterial area distension with pressure in relation to a known hydrostatic pressure. This maneuver is a form of calibration. The method also fits an exponential decay curve on the diastolic portion of the arterial distension pulse to estimate initial values of PP and distension to pressure conversion coefficients.

The Lading et al. method suffers from the following issues that impact its practical general implementations. First, before any measurement, multiple measurements of hydrostatic pressure and the level of arterial distension related to the pulse need to be performed. Second, in order for the method to be accurate, measurement requires multiple sensors, namely, a sensor to record the arterial distension pulse and an elevation sensor to record hydrostatic pressure. The method also suffers from other issues affecting its accuracy. The method requires a measurement of the amount of arterial distension related to the pulse, however, the method fails to address that many sensors signals do not measure direct arterial distension pulse but a combination of flow, pressure and volume which are all variables affecting the assumed linear relationship between arterial distension and pressure and consequently the accuracy of the estimated SP and DP.

The current invention distinguishes from the prior art as it requires a single high-fidelity, non-invasive, un-calibrated peripheral or central arterial pressure or pressure related pulse waveform to estimate SP and DP or hypertension (DP/SP) class. The invention can calculate SP and DP, or determine a hypertension (DP/SP) class, from the non-invasive waveform measurement with no requirement for maneuver or cuff NIPB calibration.

### SUMMARY OF INVENTION

In one aspect, the invention pertains to a method of non-invasively measuring a patient's systolic and diastolic blood pressure, which avoids the disadvantages facing present day brachial cuff NIBP devices operating in oscillometric mode.

To implement this aspect of the invention, an un-calibrated pulse waveform with sufficient fidelity to preserve cardiovascular features of the waveform is non-invasively sensed and recorded. The pulse waveform can be sensed at a peripheral location or a central location depending on the embodiment of the invention. The term pulse waveform is used herein to mean both pressure pulse waveforms and pressure-related pulse waveforms such as a volumetric displacement waveform from a brachial cuff. The pulse waveform can be measured using a non-invasive sensor such as a tonometer, plythsmograph, bio-impedance sensor, photodiode sensor, RF sensor or sonar Doppler sensor on a peripheral artery like a radial artery, a brachial artery, finger or a central artery such as a carotid artery. In this regard, the invention provides the capability of a cuffless solution to accurately measure SP and DP. On the other hand, the invention can also be used with a cuff to record a brachial volumetric displacement waveform.

The recorded, un-calibrated pulse waveform is then scaled such that the amplitude of the scaled waveform is a set to a fixed value. For example, the minimum of the waveform can be set to Mn=0 and the peak of the waveform can be set to Mx=100. An average waveform taken over several data cycles is desirably used as the un-calibrated waveform prior to scaling.

The scaled waveform is then calibrated based on one or more cardiovascular features in the scaled waveform. This calibration is implemented by an algorithm that accurately correlates the non-invasively recorded, un-calibrated and scaled waveform to collected data based on the cardiovascular features in the scaled waveform. In some embodiments of the invention, the algorithm correlates the waveform to invasively collected data, and in other embodiments of the invention the algorithm correlates the waveform to non-invasively collected data (e.g. collected with a conventional brachial blood pressure cuff device). Linear models like auto-regressive models or/and non-linear models like nonlinear system identification and machine learning methods like decision tree, or support vector machine are used to develop the algorithm capable of implementing the invention. The calibration is able to shift and scale the amplitude of the waveform so that the minimum of the calibrated waveform accurately estimates DP and the peak of the calibrated waveform accurately estimates SP as if DP and SP were measured directly, either invasively or non-invasively (e.g. conventional brachial blood pressure cuff device) as the case may be. Accordingly, the patient's SP is estimated as the maximum value of the calibrated waveform and the patient's DP is estimated as the minimum value of the calibrated waveform.

In another aspect, the invention pertains to a method of providing a patient's blood pressure status. More specifically, the method identifies the patient's hypertension (SP/DP) classification (e.g. Optimal, Normal, High Normal, Grade I HT, Grade II HT), again with a technique that avoids the disadvantages facing present day brachial cuff NIBP devices operating in oscillometric mode. To implement this aspect of the invention, an un-calibrated pulse waveform with sufficient fidelity to preserve cardiovascular features of the waveform is non-invasively sensed and recorded as described above. Again, the pulse waveform can be sensed at a peripheral location or a central location depending on the embodiment of the invention. The pulse waveform can be measured using a non-invasive sensor such as a tonometer, plythsmograph, bio-impedance sensor, photodiode sensor, RF sensor or sonar Doppler sensor on a peripheral artery like a radial artery, a brachial artery, a finger or a central artery like a carotid artery. This aspect of the invention similarly provides the capability of a cuffless solution, although a cuff can be used to record a brachial volumetric displacement waveform when implementing this aspect of the invention.

Again the recorded, un-calibrated pulse waveform is then scaled such that the amplitude of the scaled waveform is a set to a fixed value. For example, the minimum of the waveform can be set to Mn=0 and the peak of the waveform can be set to Mx=100. An average waveform taken over several data cycles is desirably used as the un-calibrated waveform prior to scaling.

At this point in the process, the method according to this aspect of the invention is different from the method according to the first aspect of the invention. When implementing this aspect of the invention, parameter values are determined for one or more cardiovascular features of the scaled waveform. A classification algorithm correlates the parameter values determined for one or more cardiovascular features of the scaled waveform to multiple hypertension classifications (e.g. Optimal, Normal, High Normal, Grade I HT, Grade II HT). Linear models like auto-regressive models or/and non-linear models like nonlinear system identification and machine learning methods like decision tree, or support vector machine are used to develop the classification algorithm. Accordingly, one of the multiple hypertension classifications is selected based on the parameter values of the one or more cardiovascular features determined from the scaled waveform using the classification algorithm, and the selected hypertension classification is displayed for the viewing.

The invention can be implemented using a digital signal processor and a computer with a monitor. It can also be implemented, in whole or in part, as wearable device that can continuously and accurately measure either SP and DP or a hypertension classification.

### BRIEF DESCRIPTION OF THE DRAWINGS

FIG. 1 is the schematic drawing of an implementation of one embodiment of the invention which in general consists of recording a non-invasive peripheral (e.g., brachial, radial or finger) or central (e.g., carotid), arterial pulse waveform, rescaling the waveform with set values, detecting cardiovascular related features from the scaled waveform, applying an algorithm to the values of the cardiovascular features to select which equation should be used to calculate a calibrated waveform from the scaled waveform, applying the selected equation to the scaled waveform, and a detecting the maximum and minimum of the outputted calibrated waveform as the SP and DP respectively.

FIG. 2 is the schematic drawing of an implementation of another embodiment of the invention which in general consists of recording a non-invasive peripheral (e.g., brachial, radial or finger) or central (e.g., carotid) artery pulse waveform, rescaling the waveform with set values, detecting cardiovascular related features from the scaled waveform, and applying an algorithm on the values of the cardiovascular features that determines a clinical classification for which the patient's SP and DP are expected to qualify, such as optimal, normal, high normal, and grade of hypertension.

FIG. 3 shows the form of calibration equations determined for scaled peripheral or central arterial pulse waveforms with set values Mx and Mn. The calibration equations produce a calibrated waveform where the maximum and minimum correspond accurately to directly measured SP and DP respectively.

FIG. 4 illustrates some cardiovascular related features of a non-invasive peripheral arterial pulse waveform (some of them having been detailed in U.S. Pat. No. 5,265,011), which are used when implementing the embodiments of the invention illustrated in FIGS. 1 and 2 using a non-invasive sensor to measure a pulse waveform in a peripheral artery.

FIG. 5 illustrates some cardiovascular related features of a non-invasive central artery pulse waveform (some of them having been detailed in U.S. Pat. No. 5,265,011). The cardiovascular related features are used when implementing the embodiments of the invention illustrated in FIGS. 1 and 2 using a non-invasive sensor to measure a pulse waveform in the carotid artery.

FIG. 6 shows an example of a decision tree that selects the appropriate calibration equation for applying to the scaled waveform based on the values of the cardiovascular features of the scaled waveform.

FIG. 7 shows an example of a decision tree that selects an appropriate clinical classification for which the patient's SP and DP are expected to qualify, such as optimal, normal, high normal, and grade of hypertension, based on the values of the cardiovascular features of the scaled waveform.

### DETAILED DESCRIPTION

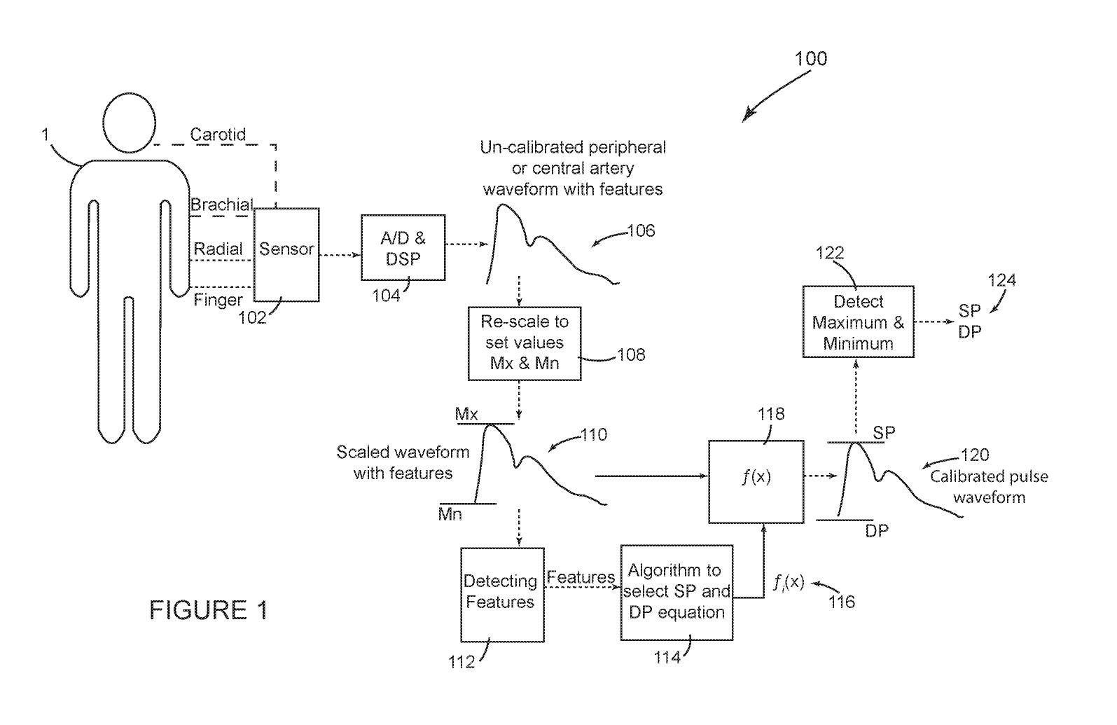

FIG. 1 shows a system 100 configured in accordance with a first embodiment of the invention. This embodiment requires a sensor 102 to non-invasively record an arterial pulse waveform. The term pulse waveform, as mentioned above, includes pressure pulse waveforms as well as other pulse waveforms such as volumetric displacement waveforms. FIG. 1 indicates that the non-invasive pulse waveform can be measured at a central location such as the carotid artery or a peripheral location such as the brachial or radial artery or in the finger. Various non-invasive sensors 102 can be used such as a tonometer, plethysmograph, bio-impedance, Doppler sensor or brachial cuff device to record non-invasive pressure or pressure related arterial pulse waveform from a peripheral artery (like finger, radial or brachial artery) or a central artery (like carotid artery).

One of the objects of the invention is to avoid measuring SP and DP with a NIBP cuff device operating in oscillometric mode; however, a cuff device can be used in accordance with the invention to capture a high-fidelity, brachial volumetric displacement waveform, as described in the Qasem U.S. Pat. No. 9,314,170, incorporated herein be reference.

It is contemplated that the sensor 102 could be a wearable sensor such as a tonometer, plythsmograph, bio-impedance, photodiode sensor, RF sensor or Doppler sensor, that records the non-invasive pressure or pressure related arterial pulse waveform from a peripheral artery or a central artery.

Through the A/D & DSP unit 104, the recorded analogue signal is converted into a digital signal and digitally processed by applying suitable high pass, low pass, band pass filters or combination of these filters in order to produce a high-fidelity, un-calibrated waveform 106 with cardiovascular related features preserved.

In another embodiment, the sensor 102 records continuous pulses for a specified amount of time (e.g., 5 or 10 seconds) and the DSP units (2) converts the string of pulses into digital data, and filters the data high pass, low pass, band pass filters or combination of these filters, and (3) then averages all the pulses to obtain a single average pulse waveform with cardiovascular related features preserved.

In one alternative, the sensor 102 can be a NIBP cuff device that measures non-invasive systolic and diastolic pressures (NISP and NIDP respectively) and records a raw oscillometric cuff waveform while the cuff is inflated to a constant pressure (below NIDP, between NIDP and NISP or above NISP). The raw signal from the NIBP cuff unit is sent to the digital signal processor 104, which filters the signal to ensure that the cardiovascular waveform features are preserved and converts the waveform to digital data for processing. As discussed above, the raw cuff waveform is processed through a high pass filter and low pass filter or a band pass filter to produce an un-calibrated brachial cuff waveform with cardiovascular related features preserved. This waveform is a brachial cuff volumetric displacement waveform, which contains and preserves the cardiovascular features present in the patient's brachial pressure waveform. The pressure of the inflated cuff will affect the shape of the recorded waveform; and therefore it is important that the cuff be inflated to a range consistent with the inflation of the cuff for the data collected to determine the calibration equations discussed below. In particular, the shape changes significantly depending on whether the cuff is inflated below the patient's DP, between DP and SP or above SP. For example, if the calibration equations are determined based on data collected with the cuff inflated below diastolic pressure for the test population, then the raw brachial (volumetric displacement) waveform should be collected with the cuff inflated below the patient's diastolic. It is preferred that the inflated cuff pressure have a 10% difference or more compared the patient's DP in order to avoid borderline effects. The same considerations apply with respect to both DP and SP in the case that the recalibration equations are determined based on data collected with the cuff inflated between DP and SP for the test population, or with respect to SP in the case that the calibration equations are determined based on data collected with the cuff inflated above SP for the test population. It is possible that a non-invasive waveform 106 captured using a pressure sensor like a tonometer may not need much filtering. On the other hand, if a brachial cuff device is used to capture the raw un-calibrated waveform, substantial filtering may be required. While the filtering of the raw cuff waveform is dependent on the particular cuff device, the cuff pressure relative to NISP or NIDP and NIBP unit used, the filtering in an exemplary embodiment uses a low pass filter with cutoff frequency between 30 to 40 Hz, and high pass filter with pass frequency between 0.7 to 1 Hz has been found suitable to capture a raw waveform in which the cardiovascular features, including the foot, first systolic peak, second systolic peak and incisura, are preserved in the data. The purpose of the low pass filter is to preserve volume, pressure or flow signal frequencies that are related to physiological function and eliminate noises related to environmental inferences such as power sources noise. The choice of the low pass cutoff frequency is based on the fact that all physiological features in pressure, volume or flow waveforms are within 25 Hz of the signal spectrum (See FIG. 26.21 in W. Nichols and M. O'Rourke, “McDonald's Blood Flow in Arteries: Theoretical, Experimental and Clinical Principles”, 5^{th} Edition). The purpose of the high pass filter is to eliminate low frequencies related to artifacts noise as a result of arm movements, breathing effect or the tube and cuff compliance in reaction to pressure. These low frequency artifacts, which cause signal baseline drift and can dampen signal shape, are usually below 1 Hz, hence the high pass filter pass frequency. Both filters, which can be implemented as a Chebyshev type filters with pass band ripple or stop band ripple of −3 dB, can be combined into one band pass filter where it pass all frequencies between 0.7 to 40 Hz.

The operations after block 104 in FIG. 1 are also preferably implemented in a digital signal processor 104, or other computing device. However, the electronic filters discussed in connection with acquiring the raw waveform can be analog or digital, or a combination of both.

Block 108 represents software that rescales the un-calibrated peripheral (or central) waveform 106 such that its maximum and minimum are set equal to pre-set scaling values Mx and Mn, which can be any number such as Mx=100 and Mn=0. The result is a scaled waveform 110 in which the cardiovascular features are preserved.

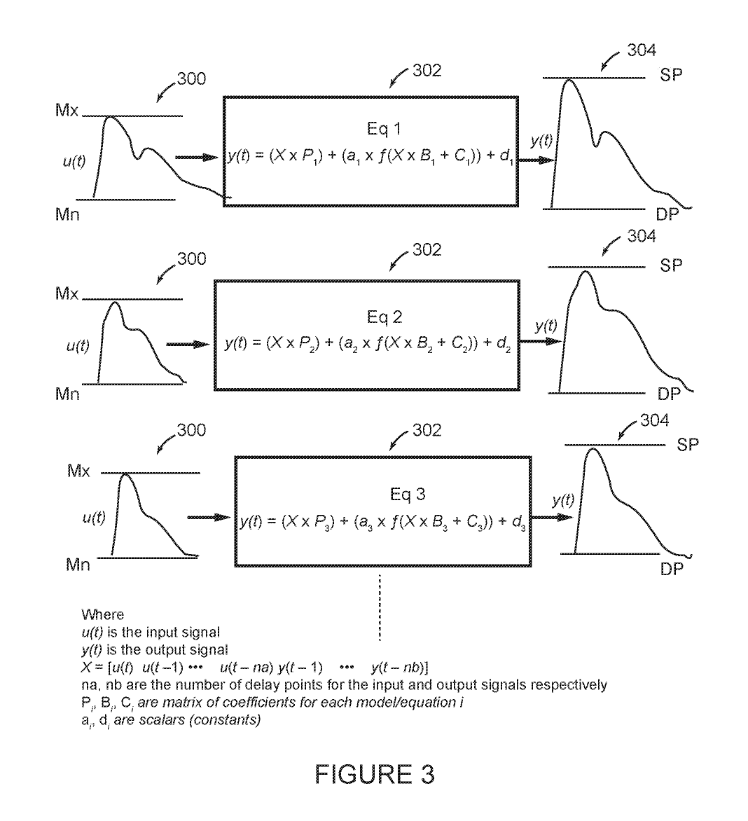

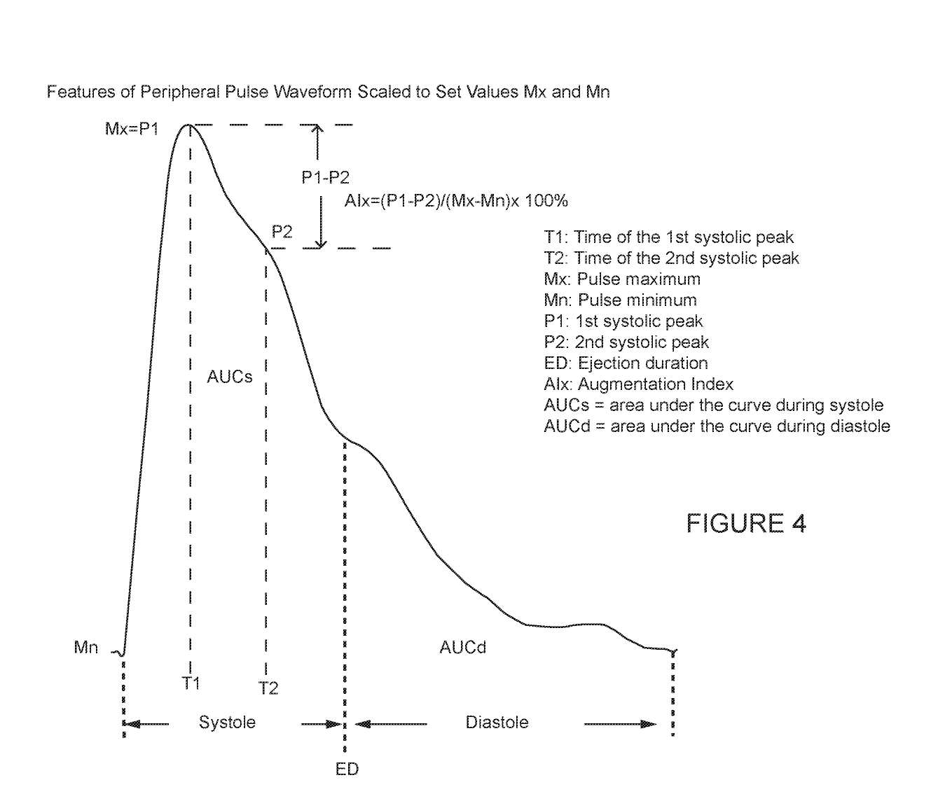

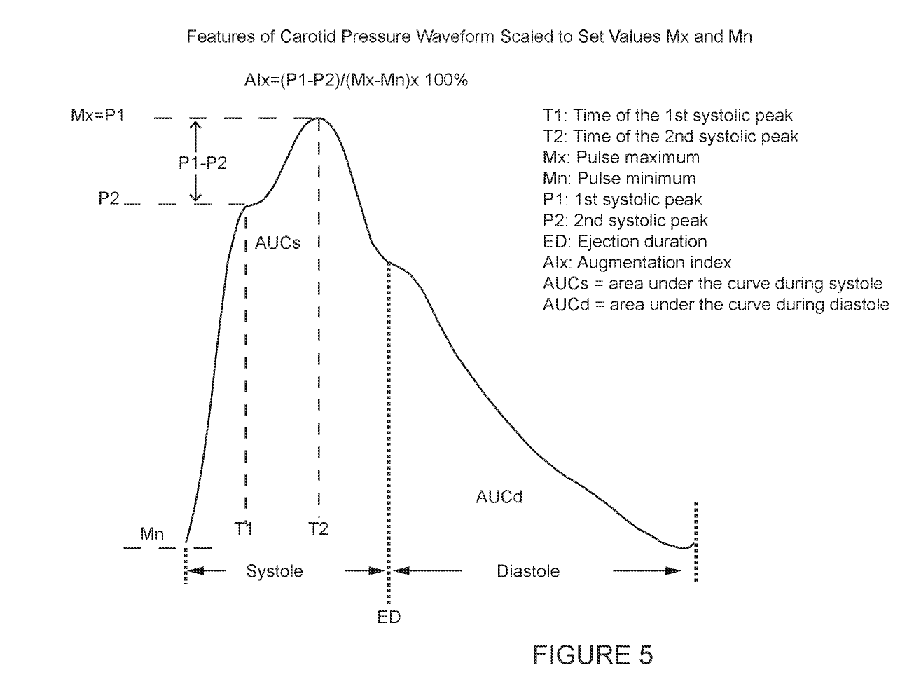

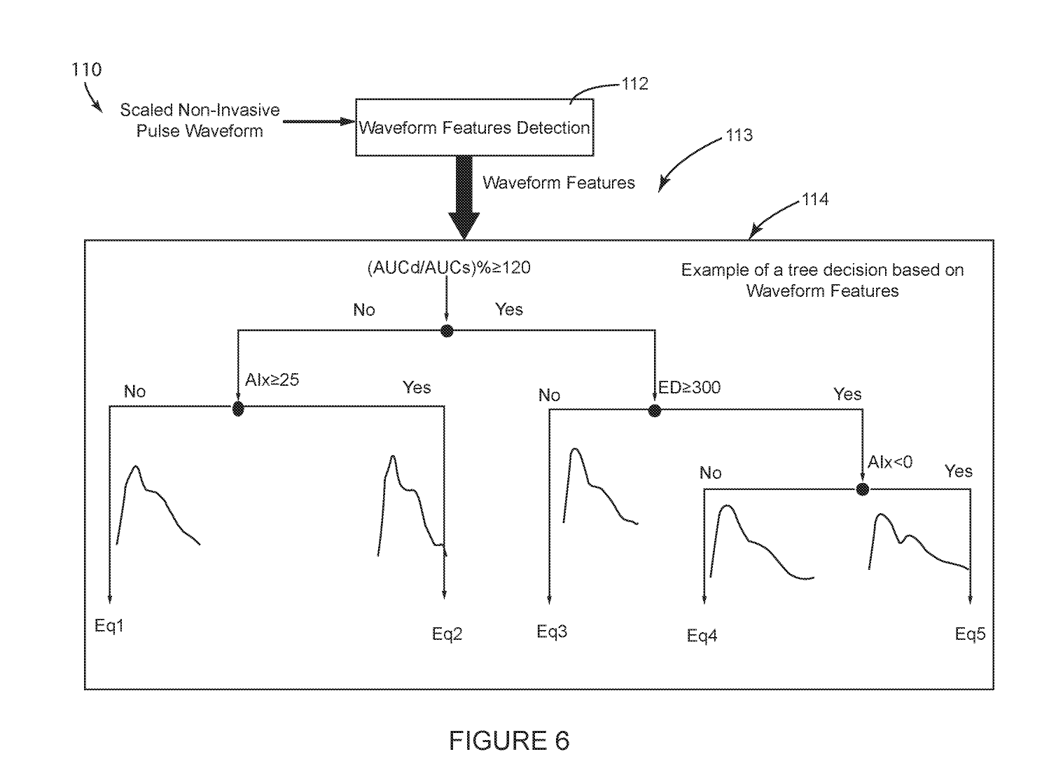

Block 112 depicts the scaled pulse waveform 110 being input for an algorithm to detect parameter values for identified cardiovascular features of the scaled waveform 110. Some of these cardiovascular features have been described in U.S. Pat. No. 5,265,011 and are described below in connection with FIG. 4 (scaled peripheral waveform) and FIG. 5 (scaled central waveform). The algorithm 112 can detect cardiovascular features using the derivative method as described in U.S. Pat. No. 5,265,011, the wavelet method, or any other suitable method. The detected features from block 112 are the input for an algorithm 114 that selects one of several calibration equations fᵢ(x) 116 to calibrate the scaled waveform resulting in a calibrated waveform 120 (peripheral or central depending whether the sensor 102 and the algorithm 114 used to detect the cardiovascular features are specific for a peripheral waveform or a central waveform). The algorithm 114 shifts and/or scales the scaled waveform 110, so that its minimum value corresponds to the patient's arterial DP and its maximum corresponds to the patient's arterial SP. The selection algorithm 114 and the calibration equations fᵢ(x) 116, as illustrated in FIGS. 6 and 3, are described in more detail below. Block 118 in FIG. 1 indicates that the selected calibrated equation fᵢ(x) 116 is applied to the scaled waveform 110 to generate the calibrated pulse waveform 120. As mentioned, the selected calibration equation 116 produces a calibrated waveform 120 where its maximum and the minimum are estimates of invasively or non-invasively measured SP and DP, respectively, for the location at which the sensor 102 measures the non-invasive waveform. Block 122 indicates that the software detects the maximum and minimum values from the calibrated waveform 120 to estimate values for SP and DP. As mentioned, the purpose of the invention is for these values of SP and DP to closely estimate the invasively or non-invasively measured SP and DP.

The SP and DP values measured using the invention, can also be used to calibrate waveforms. For example, the current method can be used with a brachial cuff to capture an un-calibrated volumetric displacement waveform, and calibrate the waveform so that its minimum accurately estimates the patient's DP and its maximum accurately estimates the patient's SP. Without the calibration error, the transfer function method can be applied, if desired, to the calibrated brachial waveform to accurately determine the central aortic waveform without significant calibration error.

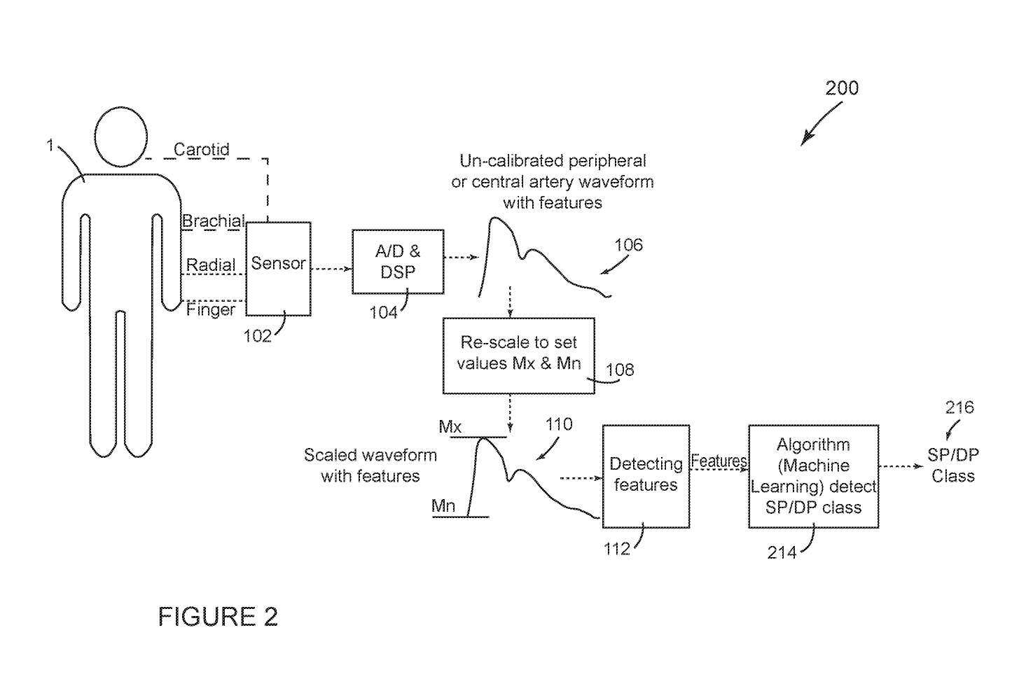

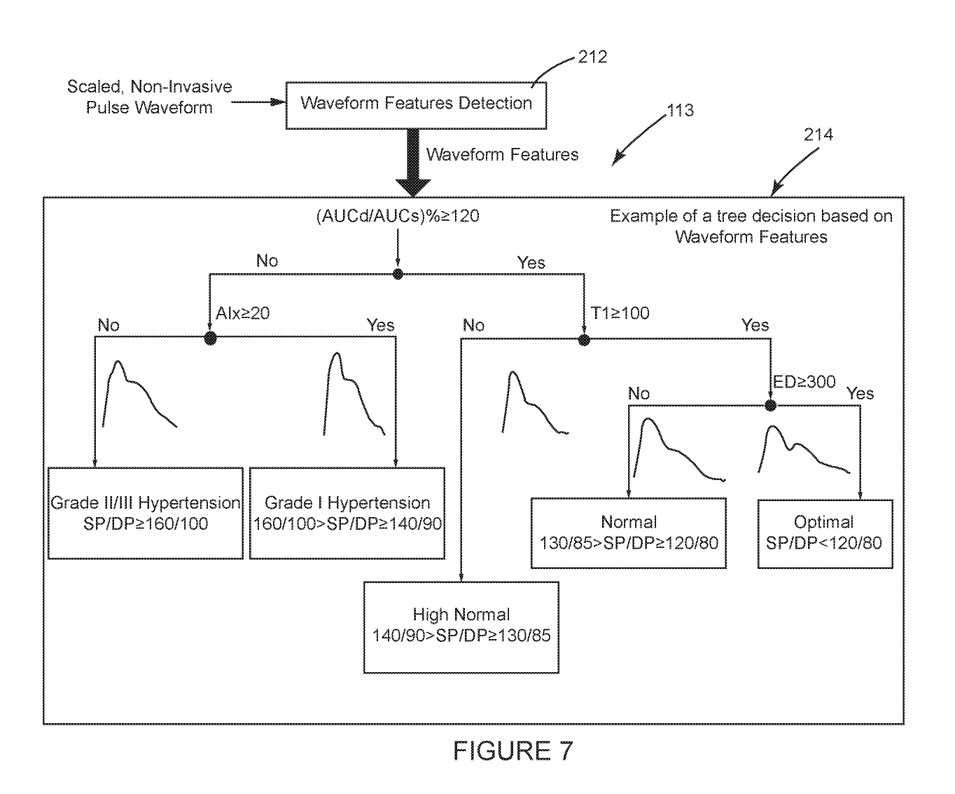

FIG. 2 shows a system 200 configured in accordance with the second embodiment of the invention. Many aspects of system 200 shown in FIG. 2 are the same or similar to system 100 shown in FIG. 1. The same reference numbers are used in FIG. 2 for components that are the same as in FIG. 1. In general, the method of operation of system 200 in FIG. 2 is similar to the operation of system 100 in FIG. 1 through the processing step identified by block 112 in both FIGS. 1 and 2, when the respective systems 100, 200 detect parameter values for cardiovascular features in the scaled waveform 110. At this point in the process, the system 200 shown in FIG. 2 deviates from the system 100 shown in FIG. 1. In FIG. 2, the detected features from block 112 are input for a classification algorithm 214 that determines a clinical classification 216 for which the patient's SP and DP are expected to qualify, such as optimal, normal, high normal, grade I hypertension and grade II hypertension based on American Heart association and European Society of Hypertension classification. (Chobanian A. et al “Seventh Report of the Joint National Committee on Prevention, Detection, Evaluation, and Treatment of High Blood Pressure” Hypertension 2003; 42:1206-1252, and Mancia G et al “The task force for the management of arterial hypertension of the European Society of Hypertension” European Heart Journal 2007; 28:1462-1536) The classification algorithm 214 is described in more detail described below with respect to FIG. 7.

The calibration equations 118 in the embodiment shown in FIG. 1 and the classification algorithm 214 shown in FIG. 2 can be determined by comparing non-invasively, un-calibrated collected data to invasively or non-invasively measured arterial pressure data. Data of un-calibrated, non-invasive peripheral or central arterial waveforms have been collected alongside recordings of invasively or non-invasively measured SP and DP values on a group representative of the general population (in term of age, height, weight, gender). Non-invasive un-calibrated arterial waveforms and invasively or non-invasively measured pressure values can be compared for measurements taken at the radial, finger, brachial and carotid arteries, respectively. The data in each case can be used to establish calibration equations (block 118) suitable to calculate SP and DP from the scaled pulse waveform 110.

Referring to FIG. 3, a method of system identification can be used to establish the coefficients for proposed calibration equations 302. In this exemplary embodiment, the form of the calibration equations is a non-linear sigmoid function, which constitutes linear and non-linear components. In general, the non-invasively un-calibrated collected waveform data is filtered and scaled, where Mx and Mn correspond to the maximum and minimum of the scaled non-invasive waveform. The scaled non-invasive un-calibrated waveform data is the input 300 for the proposed calibration equations 302. The calibrated waveform 304 for the respective artery, with its maximum and minimum values equal to (invasively or non-invasively) measured SP and DP, respectively, is the output of the proposed calibration equations 302. Given the known input 300 and output 304 from the collected data, calibration equations 302 with unknown coefficients are proposed. Then, the coefficients are estimated such that the difference between the equation output and the data collected for the blood pressure measurements is minimized. The calibration equations can theoretically be linear or non-linear or combination of both types, however, it has been found that using a non-linear component produces more accurate results.

In this example, the form of the proposed calibration equations 302, has linear and non-linear parts and can be expressed as follow:

y(t)=(X×Pᵢ)+(aᵢ×f(X×Bᵢ+Cᵢ))+dᵢ [1]

where
y(t) is the output waveform at time t

Pᵢ, Bᵢ, Cᵢ are matrices of coefficients for each equation i, and

aᵢ, dᵢ are scalars (constants).

Vector X in equation [1] is a vector of delayed input and output values which can be represented as follow:

X=[u(t)u(t−1) . . . u(t−na)y(t−1) . . . y(t−nb)] [2]

Where

u(t) is the input waveform at time t,

u(t−1) is the input waveform at time t−1,

u(t−na) is the input waveform at time t−na,

y(t−1) is the output waveform at time t−1,

y(t−nb) is the input waveform at time t−nb, and

na, nb are the number of delay points for the input and output signals respectively.

In equation [1], f( ) is a non-linear function which in this example is a sigmoid function expressed as follow:

$$f ( z ) = \frac{1}{{e}^{- z} + 1} \qquad [ 3 ]$$

To illustrate how the equation work, let's assume that na and nb are equal to 1 then vector X in equation [1] will be

X=[u(t)u(t−1)y(t−1)] [4]

Accordingly

$$\begin{matrix}{P}_{i} = [ \begin{matrix}{p}_{1} \\ {p}_{2} \\ {p}_{3}\end{matrix} ] & [ 5 ] \\ {B}_{i} = [ \begin{matrix}{b}_{1 , 1} & {b}_{1 , 2} & {b}_{1 , 3} \\ {b}_{2 , 1} & {b}_{2 , 2} & {b}_{2 , 3} \\ {b}_{3 , 1} & {b}_{3 , 2} & {b}_{3 , 3}\end{matrix} ] & [ 6 ] \\ {C}_{i} = [ {c}_{1} {c}_{2} {c}_{3} ] & [ 7 ]\end{matrix}$$

Then substituting equations [4] to [7] into equation [1], the result will be

$$y ( t ) = ( [ u ( t ) u ( t - 1 ) y ( t - 1 ) ] \times [ \begin{matrix}{p}_{1} \\ {p}_{2} \\ {p}_{3}\end{matrix} ] ) + ( {a}_{i} \times f ( [ u ( t ) u ( t - 1 ) y ( t - 1 ) ] \times [ \begin{matrix}{b}_{1 , 1} & {b}_{1 , 2} & {b}_{1 , 3} \\ {b}_{2 , 1} & {b}_{2 , 2} & {b}_{2 , 3} \\ {b}_{3 , 1} & {b}_{3 , 2} & {b}_{3 , 3}\end{matrix} ] + [ {c}_{1} {c}_{2} {c}_{3} ] ) ) + {d}_{i} \qquad [ 8 ]$$

The aim of the system identification is to estimate coefficient matrices Pᵢ, Bᵢ, Cᵢ and the constants aᵢ, dᵢ by minimizing the difference between estimated output 304 and the (invasively or non-invasively) measured pressure data.

Applying the system identification method on the (invasively or non-invasively) measured pressure data collected from a sample of the general population may for example result in five (5) different calibration equations fᵢ(x) 116 (see, FIG. 1) that can be implemented on the general population. In other words, the final form of the proposed calibration equations 302 in FIG. 3 corresponds to the calibration equations fᵢ(x) 116 programmed in to the system 100, and used in practice to detect peripheral or central SP and DP, depending on whether the system is designed to detect a peripheral waveform or a central waveform. The final form of the proposed calibration equations 302 is determined for different groupings of input 300 and output 304 waveform data, in which the groupings are based on waveform feature parameters determined by applying the system identification method. In the embodiment shown in FIG. 1, the selection algorithm 114 can be, e.g., a decision tree that determines which calibration equation fᵢ(x) 116 should be used based on waveform features.

Even though the input waveforms are scaled versions of un-calibrated, non-invasive waveforms, the method of determining the calibration equations results in the ability of the calibration equations fᵢ(x) to shift the waveform and scale the amplitude of the waveform so that its minimum correlates with data collected for the patient's (invasively or non-invasively) measured DP and its maximum correlates with data collected for the patient's (invasively or non-invasively) measured SP. In other words, using machine learning or deep learning techniques, accurate information about measured SP and DP are extracted from the shape of the patient's un-calibrated, scaled non-invasive waveform.

FIG. 4 describes cardiovascular related features of the scaled peripheral pulse waveform 110. Some of these features have been described e.g., in U.S. Pat. No. 5,265,011. Values for parameters pertaining to the features are used as inputs to the selection algorithm 114 for the embodiment shown in FIG. 1 and for the classification algorithm 214 for the embodiment shown in FIG. 2, in the case that the non-invasive waveform is a peripheral pulse waveform as distinguished from a central or carotid waveform. These features can be detected through the derivative method (as mentioned in U.S. Pat. No. 5,265,011) or any other suitable mathematical method in time or frequency like wavelet analysis. Exemplary features that can be used by the selection algorithm 114 or classification algorithm 214 include, for example, AIx, AUCs/AUCd, P1, P2, T1, T2, and ED as described in FIG. 4. Other features like heart rate, cardiac period and slope of the systolic upstroke, which also can be detected from the scaled peripheral waveform, can also be used as input to the algorithms.

FIG. 5 describes cardiovascular related features of a scaled central (e.g., carotid) pressure waveform, some of which were described in U.S. Pat. No. 5,265,011. Values for parameters pertaining to the features are used as inputs to the selection algorithm 114 for the embodiment shown in FIG. 1 and for the classification algorithm 214 for the embodiment shown in FIG. 2, in the case that the non-invasive waveform is a central or carotid pulse waveform as distinguished from a peripheral pulse waveform. These features can be detected through the derivative method (as mentioned in U.S. Pat. No. 5,265,011) or any other suitable mathematical method in time or frequency like wavelet analysis. Exemplary features that can be used by the selection algorithm 114 or classification algorithm 214 include, for example, AIx, AUCs/AUCd, T1, T2, and ED as described in FIG. 4. Other features like heart rate, cardiac period and slope of the systolic upstroke, which also can be detected from the scaled central waveform, can also be used as input to the algorithms.

The selection algorithm 114, which selects the appropriate equation to estimate SP and DP from an un-calibrated arterial waveform based on the cardiovascular related features of the scaled waveform, can be developed using different machine learning methods like decision tree, support vector machine, linear and nonlinear regression, and neural network. For the resulting algorithm 114, the waveform's features are the input while the calibration equations 116 to estimate SP and DP from the scaled, un-calibrated arterial waveform are the output. As mentioned above, this is possible because known data representing the general population that includes waveform features are used to develop to calibration equations 116 and the selection algorithm 114.

FIG. 6 illustrates one exemplary selection algorithm 114 in the form of a decision tree that is used to select a suitable calibration equation 116 based on the detected or calculated waveform features or parameters. The calibration equations 116 are labelled Eq1, Eq2, Eq3, Eq4 and Eq5 in FIG. 6. The selected calibration equation (Eq1, Eq2, Eq3, Eq4 or Eq5) is used to estimate SP and DP from scaled, un-calibrated arterial waveform based on parameter values pertaining to the waveform features. In FIG. 6, block 112 indicates that pulse waveform features 113 are detected or calculated from a scaled version of the non-invasively un-calibrated recorded pulse waveform 110. As mentioned, suitable feature detection methods, block 112, include the derivative method or other mathematical methods in time or frequency domain. The values detected or calculated pertaining to the waveform features 113 are the input to the decision tree 114, which in this example serves as the selection algorithm 114 in FIG. 1. The decision tree 114 decides which calibration equation Eq1, Eq2, Eq3, Eq4 or Eq5 to use according to the values of the detected or calculated waveform features. Specifically, in FIG. 6, one of five calibration equations (Eq1, Eq2, Eq3, Eq4 or Eq5) is selected based on values of AIx, ED, heart rate (HR) and the percentage ratio of AUCd to AUCs. The threshold values identified in FIG. 6 are illustrative and are estimated based on data analysis, although additional data collection and analysis may result in modified values. Other examples may use more waveform features with more branches in the decision tree. Also, other algorithms that correlate the waveform features with the appropriate calibration equation like support vector machine, linear and nonlinear regression, and neural network can also be used as the selection algorithm.

FIG. 7 pertains to the embodiment shown in FIG. 2, where a classification algorithm 214 is used in place of a selection algorithm 114 and calibration equations 216 as in the embodiment in FIG. 1. The classification algorithm 214 is developed to detect the hypertension (SP/DP) class (as classified by American Heart Association and European Society of Hypertension) from the scaled, un-calibrated arterial waveform 110 based on the recorded waveform cardiovascular related features. The classification algorithm 214 uses a machine learning method like decision tree, support vector machine, linear and nonlinear regression, and neural network. The waveform features are the input while the SP/DP class is the output. As mentioned above, this is possible because known data representing the general population that includes waveform features are used to develop to develop to the correlation with SP/DP classification.

FIG. 7 illustrates one exemplary classification algorithm 114 in the form of a decision tree that is used to select a suitable SP/DP classification based on parameter values detected or calculated for cardiovascular waveform features in a peripheral or central waveform. The threshold values identified in FIG. 7 are illustrative and are estimated based on data analysis, although additional data collection and analysis may result in modified values. In FIG. 7, the SP/DP classifications are: Optimal [SP/DP<120/80 mmHg], Normal [120/80≤SP/DP<130/85], High Normal [130/85≤SP/DP<140/90], Grade I Hypertension [140/90≤SP/DP<160/100] and Grade I/II hypertension [160/100≤SP/DP]. Block 212 in FIG. 7 indicates that pulse waveform features are detected from the non-invasively un-calibrated recorded, scaled waveform using detection methods like the derivative method or other suitable mathematical methods in time or frequency domain. The values for the detected waveform features 113 are the input to the decision tree 214 which according to the values of the identified waveform features selects the SP/DP class for the patient. In this example, the selection is based on values of AIx, ED, Heart rate (HR) and the percentage ratio of AUCd to AUCd. The value of the percentage ratio of AUCd to AUCd is used in the first step to determine whether the patient should be classified as having hypertension. If so the value of the augmentation index AIx is used to determine whether the hypertension is grade I or grade II. If the patient should not be classified as having hypertension, then the value of the time of the first systolic peak determines whether the patient should be classified as high normal versus normal or optimal. If the patient should be classified as normal or optimal, the value of ejection duration ED determines whether the patient should be classified normal or optimal. Other examples may use more waveform features with more branches of the tree decision. Other algorithm that correlates the waveform features with SP/DP class like support vector machine, linear and nonlinear regression, and neural network can also be used.

As mentioned, the decision trees in FIGS. 6 and 7 are meant to be illustrative. Moreover, it is expected that the structure of the decision tree my need to be more complicated than that shown in FIGS. 6 and 7 for the systems to accurately estimate invasive SP and DP, or hypertension classification, respectively.

What is claimed is:

1. A method of non-invasively measuring a patient's systolic and diastolic blood pressure comprising the steps of:
non-invasively sensing and recording an un-calibrated pulse waveform with sufficient fidelity to preserve cardiovascular features of the waveform;
scaling the recorded, un-calibrated pulse waveform such that the amplitude of the scaled waveform is a set to a fixed value;
calibrating the scaled waveform based on one or more cardiovascular features in the scaled waveform;
estimating the patient's systolic peripheral blood pressure as the maximum value of the calibrated waveform and estimating the patient's peripheral diastolic blood pressure as the minimum value of the calibrated waveform.

2. The method as recited in claim 1 further comprising the steps of:
determining one or more parameters pertaining to the cardiovascular features of the scaled waveform;
providing multiple calibration equations;
and selecting one of the multiple recalibration equations based on the one or more cardiovascular features determined from the scaled waveform.

3. The method as recited in claim 2 wherein the determined one or more cardiovascular parameters include augmentation index, ejection duration, and the ratio of area under the curve during diastole divided by the area under the curve during systole.

4. The method as recited in claim 2 wherein the calibration equation is selected using a decision tree.

5. The invention as recited in claim 2 wherein the multiple calibration equations are determined by comparing data collected for a sampling of the general population comparing scaled, un-calibrated, non-invasive waveform data to invasively measured waveform data including systolic and diastolic blood pressure data.

6. The invention as recited in claim 2 wherein the multiple calibration equations are determined by comparing data collected for a sampling of the general population comparing scaled, un-calibrated, non-invasive waveform data to non-invasively measured waveform data including systolic and diastolic blood pressure data.

7. The method as recited in claim 2 wherein the multiple calibration equations include linear components and non-linear components.

8. The method as recited in claim 7 wherein each of the multiple calibration equations as the following form:
y(t)=([u(t)u(t−1) . . . u(t−na)y(t−1) . . . y(t−nb)]×Pᵢ)+(aᵢ×f([u(t)u(t−1) . . . u(t−na)y(t−1) . . . y(t−nb)]×Bᵢ+Cᵢ))
where
y(t) is the output waveform at time t
Pᵢ, is na+nb+1 by 1 matrix of coefficients for recalibration equation i
Bᵢ, is na+nb+1 by na+nb+1 square matrix of coefficients for recalibration equation i
Cᵢ is na+nb+1 by 1 matrix of coefficients for recalibration equation i
na, nb are the number of delay points for the input and output signals respectively,
aᵢ, dᵢ are scalars (constants) for recalibration equation i
u(t) is the input waveform at time t,
u(t−1) is the input waveform at time t−1,
u(t−na) is the input waveform at time t−na,
y(t−1) is the output waveform at time t−1,
y(t−nb) is the input waveform at time t−nb, and
and f( ) is a non-linear sigmoid function expressed as follows:
$$f ( z ) = \frac{1}{{e}^{- z} + 1} .$$

9. The method as recited in claim 1 wherein the un-calibrated pulse waveform that is non-invasively sensed and recorded is a peripheral waveform.

10. The method as recited in claim 1 wherein the un-calibrated pulse waveform that is non-invasively sensed and recorded is a brachial cuff volumetric displacement waveform.

11. The method as recited in claim 1 wherein the un-calibrated pulse waveform that is non-invasively sensed and recorded is a carotid waveform.

12. The method as recited in claim 1 wherein the step of non-invasively sensing and recording an un-calibrated pulse waveform with sufficient fidelity to preserve cardiovascular features of the waveform includes filtering of a raw signal from a sensor.

13. A method of providing a patient's blood pressure status comprising the steps of:
non-invasively sensing and recording an un-calibrated pulse waveform with sufficient fidelity to preserve cardiovascular features of the waveform;
determining parameter values for one or more cardiovascular features of the scaled waveform;
providing multiple hypertension classifications;
and selecting one of the multiple hypertension classifications based on the parameter values of the one or more cardiovascular features determined from the scaled waveform; and
displaying the selected hypertension classification.

14. The method as recited in claim 13 wherein the determined one or more cardiovascular parameters include augmentation index, ejection duration, and the ratio of area under the curve during diastole divided by the area under the curve during systole.

15. The method as recited in claim 13 wherein the hypertension classification is selected using a decision tree.

16. The invention as recited in claim 13 wherein an algorithm that selects the hypertension classification for the patient is established by comparing data collected for a sampling of the general population comparing scaled, un-calibrated, non-invasive waveform data to invasively measured waveform data including systolic and diastolic blood pressure data.

17. The invention as recited in claim 13 wherein an algorithm that selects the hypertension classification for the patient is established by comparing data collected for a sampling of the general population comparing scaled, un-calibrated, non-invasive waveform data to non-invasively measured waveform data including systolic and diastolic blood pressure data.

18. The method as recited in claim 13 wherein the un-calibrated pulse waveform that is non-invasively sensed and recorded is a peripheral waveform.

19. The method as recited in claim 13 wherein the un-calibrated pulse waveform that is non-invasively sensed and recorded is a brachial cuff volumetric displacement waveform.

20. The method as recited in claim 13 wherein the un-calibrated pulse waveform that is non-invasively sensed and recorded is a carotid waveform.

21. The method as recited in claim 13 wherein the step of non-invasively sensing and recording an un-calibrated pulse waveform with sufficient fidelity to preserve cardiovascular features of the waveform includes filtering of a raw signal from a sensor.
